(19) United States
(12) Patent Application Publication
(10) Pub. No.: US 2024/0268972 A1
(43) Pub. Date: Aug. 15, 2024

(54) FUSION CAGE

(71) Applicant: Zhisu Health Technology (Jiaxing) Co., Ltd., Jiaxing (CN)

(72) Inventors: Jing ZHANG, Jiaxing (CN); Kai XU, Jiaxing (CN); Lu SUN, Jiaxing (CN); Hao LIU, Jiaxing (CN); Xuewei MA, Jiaxing (CN)

(21) Appl. No.: 18/444,692

(22) Filed: Feb. 18, 2024

Related U.S. Application Data
(63) Continuation of application No. PCT/CN2022/081247, filed on Mar. 16, 2022.

(30) Foreign Application Priority Data
Sep. 13, 2021 (CN) 202111067197.7

(51) Int. Cl. A61F 2/44; A61F 2/28

(52) U.S. Cl. CPC A61F 2/447; A61F 2002/2835

(57) ABSTRACT

The present invention provides a fusion cage. The fusion cage includes a first end surface, designed to conform to and support human tissue. The first end surface is provided with a first lattice structure extending into the interior of the fusion cage. The first lattice structure is intended to conform to and support human tissue, providing a pathway for bone ingrowth into the interior of the fusion cage. A second lattice structure is provided within the fusion cage, serving to reduce the overall product's elastic modulus and provide space for bone ingrowth. The primary purpose of the first lattice structure is to offer a space for the growth of human tissue, and the combined action of both lattice structures at two locations helps address the technical challenge of prior art being unable to simultaneously meet the matching bone elastic modulus and aid in the growth of human tissue.

### TECHNICAL FILED

The present invention relates to the field of medical devices, particularly to a fusion cage.

### BACKGROUND

Fusion cage is an important medical devices. For instance, lumbar intervertebral fusion cages, among others, are employed in the treatment of discogenic low back pain and various causes of lumbar spondylolisthesis. It is also used for orthopedic applications such as spinal canal decompression and repositioning fixation. However, existing intervertebral fusion cages often adopt a solid structure or lattice structure distributed on the lateral side, resulting in a higher elastic modulus, susceptibility to stress shielding, and poor bone ingrowth effects. The design of lattice structures is intended to enhance bone ingrowth. Therefore, there is an urgent need for a fusion cage capable of simultaneously meeting the requirements of matching bone's elastic modulus and promoting the growth of human tissues. The current technology lacks a solution that can address both the matching of bone elastic modulus and assisting in the growth of human tissues simultaneously.

### SUMMARY

The present application discloses a fusion cage including a first end surface designed to conform to and support human tissues. The first end surface is provided with a first lattice structure being extended into an interior of the fusion cage.

The first lattice structure is intended to conform to and support human tissues while providing the fusion cage with certain elastic modulus.

A second lattice structure is provided within the fusion cage. The second lattice structure is configured for providing the fusion cage with a designated elastic modulus.

In an alternative embodiment, the first end surface is provided with at least one inclined surface pillar.

The inclined surface pillar is a strip-like structure, with both ends spanning the first lattice structure and connecting to the first end surface.

In another alternative embodiment, the fusion cage is a pillar-like structure, with one end of the pillar-like structure protruding outward in a tapered structure.

The first end surface is positioned on a side of the pillar-like structure and is either arch-shaped or flat surface with a curvature.

The inclined surface pillar has an inclined surface, with the inclined surface oriented from high to low towards the tapered structure.

In another alternative embodiment, the at least one inclined surface pillar consists of multiple inclined surface pillars.

The multiple inclined surface pillars are spaced apart from one end to the other end of the pillar-like structure.

In another alternative embodiment, a top of the tapered structure is smoothly connected to the first end surface through a curved surface.

In another alternative embodiment, a side of the fusion cage also has a second end surface, and the second end surface is provided with a second lattice structure being extended into at least one region inside the fusion cage.

In another alternative embodiment, the at least one region includes a first region and a second region.

The first region and the second region are spaced apart.

In another alternative embodiment, A central portion between the two ends of the fusion cage forms a cuboid structure with four side surfaces, where two of the side surfaces are adjacent to the first end surface and the second end surface.

The first lattice structure is interfaced with the second lattice structure.

In another alternative embodiment, a central position of the first end surface is provided with a bone graft window, which penetrates to the opposite side of the first end surface.

In another alternative embodiment, the other end of the fusion cage is provided with an instrument thread hole.

### BRIEF DESCRIPTION OF THE DRAWINGS

FIG. 1 is a schematic diagram of the external structure of the fusion cage in one embodiment of the present invention.

FIG. 2 is a schematic diagram of the side view of the fusion cage in one embodiment of the present invention.

FIG. 3 is a schematic diagram of the top view of the fusion cage in one embodiment of the present invention.

FIG. 4 is a schematic diagram of the rear view of the fusion cage in one embodiment of the present invention.

### CHARACTER REFERENCES IN THE DRAWINGS

11, First end surface
12, First lattice structure
13, Second lattice structure
131, First region
132, Second region
14, Inclined surface pillar
141, Inclined surface
15, Tapered structure
151, Curved surface
16, Second end surface
101, Side surface
17, Bone graft window
18, Instrument thread hole

### DETAILED DESCRIPTION OF THE EMBODIMENTS

Currently, the elastic modulus and bone ingrowth effectiveness of fusion cages have been key aspects of product development. In prior art, fusion cages encompass a variety of types, such as lumbar intervertebral fusion cages. For illustration purposes, we will use lumbar intervertebral fusion cages as an example. It is important to note that lumbar intervertebral fusion cages are used merely for better elucidating this application and do not represent specific limitations thereof. After implantation into the human body, lumbar intervertebral fusion cages invariably have at least one surface in contact with the human body, typically two surfaces in contact with human tissues, to support and facilitate the growth of human tissues. Besides the mentioned issues, fusion cages also incorporate lattice structures, primarily aiming to provide the device with an elastic modulus to overcome clinical problems like stress shielding. In the existing technology, intervertebral fusion cages often adopt a solid structure or have lattice structures distributed on the lateral side, resulting in a high elastic modulus, susceptibility to stress shielding, and suboptimal bone ingrowth. There is an urgent need for a fusion cage to address the technical challenge of current technologies failing to simultaneously meet the requirements of matching bone elasticity and assisting in the growth of human tissues.

Now, with reference to the drawings in the embodiments of this application, a clear and comprehensive description of the technical solution in these embodiments will be provided. It is evident that the described embodiments are only a part of the embodiments of this application, not all of them. Based on the embodiments in this application, all other embodiments obtained by those skilled in the art without creative labor are within the scope of protection of this application.

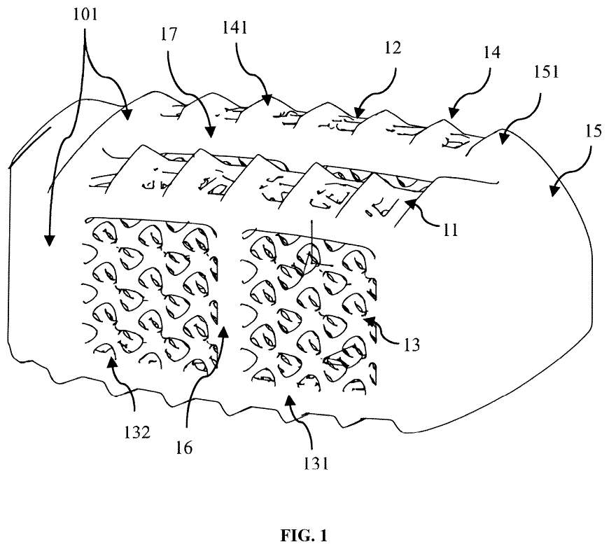

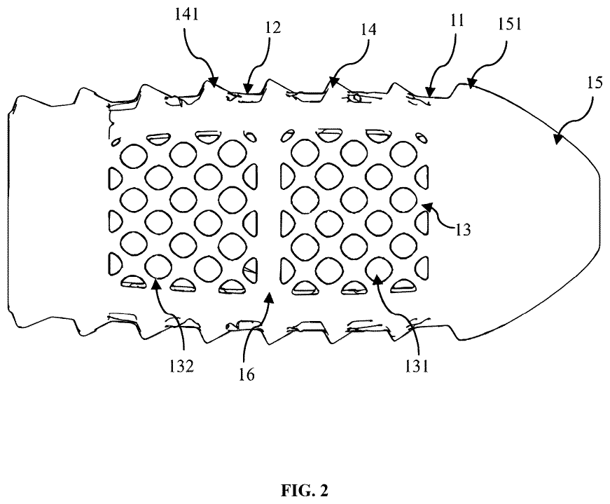

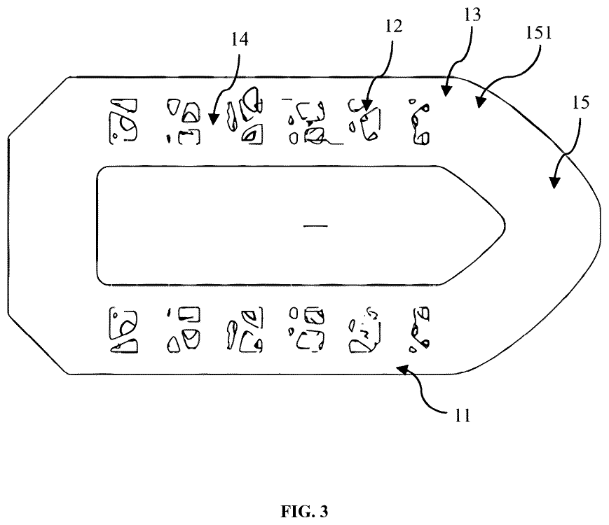

FIG. 1 is a schematic diagram illustrating the external overall structure of the fusion cage in one embodiment of the present invention. FIG. 2 is a schematic diagram of side view of the fusion cage in another embodiment of the present invention. FIG. 3 is a schematic diagram of top view the fusion cage in another embodiment of the present invention. As shown in FIGS. 1, 2, and 3, the present application provides a fusion cage having a first end surface 11. The first end surface 11 is designed to conform to and support human tissues, and it is provided with a first lattice structure 12 being extended into an interior of the fusion cage. The first lattice structure 12 can be a random or regular lattice structure.

The first lattice structure 12 is intended to conform to and support human tissues while providing the fusion cage with an elastic modulus. The distinctive feature of the first lattice structure 12 is its ability to provide a structure with elastic modulus that matches human bones. Additionally, during use, the first end surface 11 conforms to human tissues, and the first lattice structure 12 provides a space for the growth of human tissues. In other words, it offers a structure with elastic modulus that matches the bones and allows human tissues to grow through the first lattice structure 12, aiding in solving the technical problem of prior art failing to simultaneously meet the requirements of matching bone elasticity and assisting in the growth of human tissues.

A second lattice structure 13 is provided within the fusion cage. The second lattice structure 13 is intended to provide the fusion cage with a designated elastic modulus.

It should be noted that both the first lattice structure and the second lattice structure may adopt a porous structure. The porous structure is a truss structure with spatial pores constructed through struts. The preferred range of porosity is 60-90%, pore size is 250-1000 μm, and rod diameter is 100-1000 μm. Different combinations of porosity, pore size, and rod diameter can result in different bone elastic moduli.

In an alternative embodiment, at least one inclined surface pillar 14 is arranged on the first end surface 11.

The inclined surface pillar 14 is a strip-like structure, with both ends spanning the first lattice structure 12 and being connected to the first end surface 11.

In this embodiment, to enhance the strength of the first lattice structure 12, as it also needs to support human tissues, inclined surface pillars 14 are arranged on the first end surface 11. The function of the inclined surface pillars 14 is similar to that of reinforcing ribs. The inclined surface pillars 14 may be integrally formed on the first end surface 11 to enhance the overall strength of the first lattice structure 12 region. Moreover, since the first end surface 11 is also used to support human tissues, the inclined surface pillars 14 may also be used to lift the lumbar vertebrae, preventing movement after the fusion cage is implanted.

In an optional embodiment, the fusion cage is a pillar-like structure, with one end of the pillar-like structure protruding outward in a tapered structure 15.

The first end surface 11 is positioned on the side of the pillar-like structure, and the first end surface 11 is an arc-shaped surface or a flat surface with a curvature.

The inclined surface pillars 14 have inclined surfaces 141, with the inclined surfaces 141 oriented from high to low towards the tapered structure 15.

In this embodiment, further details are provided regarding the structure of the inclined surface pillars 14. The fusion cage, as a whole, forms a pillar-like structure, with one end being a tapered structure 15. The tapered structure 15 is designed for clinical use, enabling the fusion cage to be inserted between two vertebrae. The inclined surfaces 141 of the inclined surface pillars 14 face towards one end of the tapered structure 15. When the tapered structure 15 is pushed between two vertebrae, the fusion cage continues to slide between them. As the inclined surface pillars 14 enter between the two vertebrae, the gradually rising slope allows the vertebrae to smoothly slide into place.

In an optional embodiment, at least one inclined surface pillar 14 consists of multiple inclined surface pillar.

The multiple inclined surface pillars 14 are spaced apart from one end to the other end of the pillar-like structure.

This embodiment provides a specific structure where multiple inclined surface pillars 14 reinforce the fusion cage. Since the first lattice structure 12 between the two ends of the fusion cage may have a relatively long length, reinforcement can be achieved by using the multiple inclined surface pillars 14.

In an optional embodiment, the top of the tapered structure 15 is smoothly connected to the first end surface 11 through a curved surface 151.

This embodiment provides a specific structure between the tapered structure 15 and the first end surface 11. As the first end surface 11 gradually slides between the upper and lower vertebrae, a relatively smooth curved surface 151 is needed to facilitate the overall insertion of the fusion cage.

In an optional embodiment, the side of the fusion cage also has a second end surface 16, and the second end surface 16 is provided with a second lattice structure 13 being extended into at least one region within the fusion cage.

This embodiment provides a specific structure of how the second lattice structure 13 is arranged on the fusion cage.

In an optional embodiment, the at least one region includes a first region 131 and a second region 132.

The first region 131 and the second region 132 are spaced apart.

This embodiment provides a specific arrangement of the second lattice structure 13. Due to the spaced arrangement of the first region 131 and the second region 132, the portion between the first region 131 and the second region 132 remains a solid structure, enhancing strength.

In an optional embodiment, the central portion between the two ends of the fusion cage forms a cuboid structure, and the cuboid structure has four side surfaces 101, where two of the side surfaces 101 are adjacent to the first end surface 11 and the second end surface 16.

The first lattice structure 12 in interfaced with the second lattice structure 13.

This embodiment provides a specific overall structure of the fusion cage. The central portion of the fusion cage is a cuboid structure with four side surfaces 101, where one side surface 101 is the first end surface 11, and the other side surface 101 is the second end surface 16. The first end surface 11 and the second end surface 16 are adjacent. The structure on the side of the first end surface 11 opposite to the first end surface 11 can be completely symmetrical, and similarly, the structure on the side of the second end surface 16 opposite to the second end surface 16 can be completely symmetrical.

In an optional embodiment, the central position of the first end surface 11 is provided with a bone graft window 17, which penetrates to the opposite side of the first end surface 11.

This embodiment provides a specific structure with a bone graft window 17 for providing space for the growth of human tissues. Meanwhile, the fusion cage can also be designed without a bone graft window 17, and the first lattice structure 12 and the second lattice structure 13 are extended to the central region of the fusion cage, with the inclined surface pillars 14 passing through both ends of the fusion cage.

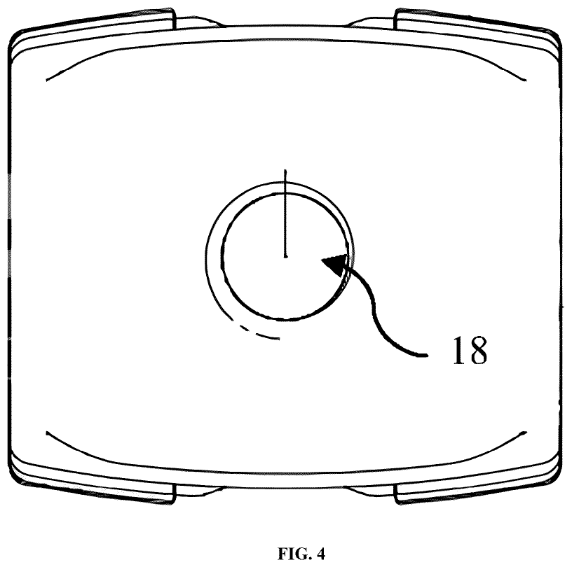

FIG. 4 is a schematic diagram of rear view of the fusion cage structure in another embodiment of the present invention. In an optional embodiment, the other end of the fusion cage is provided with an instrument thread hole 18.

In this embodiment, a specific structure is provided where the fusion cage is provided with the instrument screw hole 18. The instrument thread hole 18 is configured for inserting tools during clinical surgery.

Those skilled in the art can understand that various features disclosed in the various embodiments and/or claims can be combined and/or combined in various ways, even if such combinations or combinations are not explicitly disclosed in this application. In particular, without departing from the spirit and teachings of this application, the features recorded in various embodiments and/or claims of this application can be combined and/or combined in various ways, and all such combinations and/or combinations fall within the scope of this application.

Specific embodiments have been used to illustrate the principles and embodiments of the present invention, and the description of the above embodiments is only intended to help understand the method and core ideas of the present invention and is not intended to limit this application. For those skilled in the art, various changes, modifications, improvements, etc. can be made to the specific embodiments and applications within the spirit and principles of the present invention, and all such modifications, changes, improvements, etc. should be included in the protection scope of this application.

What is claimed is:

1. A fusion cage, characterized in that the fusion cage comprises a first end surface (11) designed to conform to and support human tissues; the first end surface (11) is provided with a first lattice structure (12) being extended into an interior of the fusion cage;
the first lattice structure (12) is intended to conform to and support human tissues while providing the fusion cage with certain elastic modulus;
a second lattice structure (13) is provided within the fusion cage; the second lattice structure (13) is configured for providing the fusion cage with a designated elastic modulus.

2. The fusion cage according to claim 1, characterized in that a side of the fusion cage further comprises a second end surface (16), and the second end surface (16) is provided with the second lattice structure (13) being extended into at least one region within the fusion cage.

3. The fusion cage according to claim 2, characterized in that the at least one region comprises a first region (131) and a second region (132);
the first region (131) and the second region (132) are spaced apart.

4. The fusion cage according to claim 1, characterized in that the first end surface (11) is provided with at least one inclined surface pillars (14);
the inclined surface pillar (14) is a strip-like structure, and both ends of the strip-like structure span the first lattice structure (12) and are connected to the first end surface (11).

5. The fusion cage according to claim 4, characterized in that the fusion cage is a pillar-like structure, with one end of the pillar-like structure protruding outward in a tapered structure (15);
the first end surface (11) is located on the side of the pillar-like structure, and the first end surface (11) is an arc-shaped surface or a flat surface with a curvature;
the inclined surface pillar (14) has an inclined surface (141), and the inclined surface (141) is arranged from high to low towards the tapered structure (14).

6. The fusion cage according to claim 5, characterized in that at least one inclined surface pillar (14) is multiple inclined surface pillars;
the multiple inclined surface pillars (14) are spaced apart from one end to the other end of the pillar-like structure.

7. The fusion cage according to claim 6, characterized in that A top of the tapered structure (15) is smoothly connected to the first end surface (11) through an arc surface (151).

8. The fusion cage according to claim 7, characterized in that a middle portion between the two ends of the fusion cage forms a cuboid structure, and the cuboid structure has four side surfaces (101), where two of the side surfaces (101) are adjacent to the first end surface (11) and the second end surface (16);
the first lattice structure (12) is interfaced with the second lattice structure (13).

9. The fusion cage according to claim 7, characterized in that a central position of the first end surface (11) is provided with a bone graft window (17), and the bone graft window (17) penetrates to one side of the first end surface (11).

10. The fusion cage according to claim 9, characterized in that the other end of the fusion cage is provided with an instrument thread hole (18).
